(19) United States
(12) Patent Application Publication
(10) Pub. No.: US 2004/0011638 A1
(43) Pub. Date: Jan. 22, 2004

(54) Method for stabilizing acrylic monomers

(76) Inventor: Stephane Lepizzera, Saint avold (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/381,795

(22) PCT Filed: Sep. 25, 2001
(86) PCT No.: PCT/FR01/02965

(30) Foreign Application Priority Data
Sep. 29, 2000 (FR) 00/12422

(51) Int. Cl. B01D 3/34; C07C 51/42

(52) U.S. Cl. 203/6; 203/49; 203/60; 203/62; 203/63; 562/600

(57) ABSTRACT

The invention concerns a method for stabilising acrylic monomers in a distillation column, comprising the following steps: adding at least a stabilising agent for acrylic monomers having a total concentration in the liquid phase ranging between 1 ppm and 5000 ppm; injecting oxygen in the distillation column with a O₂/organic vapour mol ratio ranging between 0.01% and 1%; adding a metal sequestering agent having a concentration in the liquid phase ranging between 0.1 and 1000 ppm.

The subject of the present invention is a method for stabilizing acrylic monomers, in particular acrylic acid, using stabilizers containing a metal sequestering agent.

One of the problems during the purification of acrylic monomers results from the fact that the acrylic monomer easily polymerizes when it is distilled. The formation of insoluble polymers in the industrial distillation equipment then causes blockages which require stopping the plant and cleaning it.

Another problem linked to the purification of acrylic monomers results from the corrosive nature of these products.

Now, the corrosion of stainless steel constituting industrial plants causes additional costs due to the maintenance of industrial tools, or even to the replacement of the plants.

This problem is particularly critical in distillation columns which constitute expensive installations and which often contain stainless steel components. Now, stainless steel is attacked by acrylic monomers and thus undergoes rapid degradation.

To overcome these disadvantages, various types of stabilizing molecules are used.

There are known, for example, phenolic derivatives such as hydroquinone and thiazine derivatives such as phenothiazine.

It is in addition known to use metal sequestrants to enhance the stability of (meth)acrylic monomers.

Thus, patent JP 05320205 describes the use of a nitroxide derivative with a metal sequestering agent having a stability constant greater than 10 for Fe complexes under acidic conditions to enhance the stability of (meth)acrylic monomers.

This document nevertheless describes only the use of a single stabilizer and of one metal sequestrant. It does not mention a beneficial effect of using a metal sequestrant on the corrosion of stainless steel.

Moreover, patent JP 05295011 describes the use of one or more phenothiazine (PTZ), aromatic amine or phenolic compounds with a sequestering agent having a stability constant greater than 10 for the Fe complexes under acidic conditions.

The document nevertheless does not mention a synergistic effect due to the use of several stabilizers in combination with a metal sequestrant. In addition, no favorable effect of the use of a metal sequestrant on corrosion is suggested.

In addition, polymerization inhibitors comprising one or more stabilizers and oxygen are known from EP 048 51 69.

However, these known stabilizers do not completely prevent the formation of polymers during the steps for purifying acrylic monomers.

There is still therefore a strong industrial need for mixtures of stabilizers with enhanced stabilizing efficiencies.

The problem which the invention proposes to solve is therefore to provide a method for stabilizing acrylic monomers having enhanced stabilizing efficiencies and making it possible, at the same time, to reduce the corrosion caused by acrylic monomers.

The subject of the present invention is therefore to provide a method for stabilizing acrylic monomers, in particular in a distillation column.

The method for stabilizing acrylic monomers in a distillation column according to the invention comprises the steps of adding at least one stabilizer of acrylic monomers in a total concentration in the liquid phase of between 1 ppm and 5000 ppm, of injecting oxygen into the distillation column with an O₂/organic vapor mol ratio of between 0.01% and 1%, and of adding a metal sequestrant in a concentration in the liquid phase of between 0.1 ppm and 1000 ppm.

Preferably, the stabilizer is chosen from phenolic derivatives, thiazines, transition metal salts and nitroxide derivatives.

The stabilizer is advantageously chosen from hydroquinone, hydroquinone methyl ether, phenothiazine, methylene blue, copper dibutyldithiocarbamate, manganese acetate, 2,2,6,6-tetramethyl-4-acetoxypiperidine-oxyl, 2,2,6,6-tetramethyl-4-hydroxypiperidine-oxyl, 2,2,6,6-tetramethyl-4-methoxypiperidine-oxyl and mixtures thereof.

The concentration of the stabilizer in the liquid phase is preferably between 5 ppm and 3000 ppm, preferably between 10 and 1000 ppm.

The mol ratio between the injected oxygen and the organic vapor is preferably between 0.05% and 0.5%, preferably between 0.1 and 0.25%.

The metal sequestering agent is advantageously chosen from tetraethylenediaminetetraaectic acid (EDTA), trans-1,2-cyclohexanediaminetetraacetic acid (CYDTA), diethylenetriaminepentaacetic acid (DTPA), the pentasodium salt of diethylenetriaminepentaacetic acid (Na₅DTPA), and mixtures thereof.

The metal sequestrant is present in the liquid phase preferably in a concentration of between 0.5 ppm and 500 ppm, in particular between 5 ppm and 100 ppm.

Advantageously, the acrylic monomer is chosen from acrylic acid, methacrylic acid, acrylates, methacrylates, acrylonitrile, or mixtures thereof.

According to one embodiment, at least two stabilizers are used.

Among the combinations of stabilizers, the following mixtures are particularly preferred: PTZ/HQ/Na₅DTPA, CB/HA/Na₅DTPA, CB/PTZ/HQ/Na₅DTPA, CB/PTZ/HQ/Na₅DTPA and 4-OH-TEMPO/HQME/Na₅DTPA.

Indeed, it was discovered that free radicals play an important role in the polymerization of acrylic monomers. It appears that these free radicals are generated by thermal cracking of heat-sensitive species such as peroxides. It is also possible that some oxidation-reduction reactions promote the generation of free radicals. These two processes can also occur simultaneously.

It is known that metals facilitate oxidation-reduction reactions. Such an effect is for example known for reducing the temperature for generating free radicals from the persulfate/metabisulfite system by adding iron sulfate.

Trials for stabilizing acrylic monomers in the presence of a stabilizer and a sequestering agent then made it possible to observe in addition to a synergistic effect between the stabilizer and the sequestering agent at the level of the stabilization of acrylic monomers, a reduction of corrosion.

Thus, the problems stated are solved according to the invention by adding a stabilizer and a metal sequestrant, and by injecting oxygen into the distillation column.

Indeed, it was discovered that the stabilizers mentioned show, as a mixture, a synergistic effect exceeding what it would be expected to obtain by the addition of the effects due to the individual constituents.

In addition, the method according to the present invention makes it possible to substantially improve the duration of operation of a column for distillating a stream based on acrylic monomers and to substantially reduce the corrosion of industrial tools made of stainless steel.

The expression “acrylic monomers” is understood to mean, in the present text, acrylic acid, methacrylic acid, acrylates, methacrylates and acrylonitrile, and mixtures thereof.

Among the molecules appropriate for stabilizing acrylic monomers in the context of the invention, alone or as a mixture, there may be mentioned phenolic derivatives such as, for example, hydroquinone, p-methoxyphenol, cresol, phenol, hydroquinone methyl ether, and 2,5-butyl-1-hydroxytoluene.

Thiazine derivatives, such as for example phenothiazine or methylene blue, and substituted paraphenylenediamines may also be used.

Moreover, transition metal salts such as for example copper dimethyldithiocarbamate, copper diethyldithiocarbamate, copper dibutyldithiocarbamate or the corresponding manganese salts, and manganese acetate, are also appropriate.

The use of a mixture of at least two stabilizers is preferred.

Finally, nitroxide derivatives such as, for example, 2,2,6,6-tetramethyl-4-acetoxypiperidine-oxyl, 2,2,6,6-tetramethyl-4-hydroxypiperidine-oxyl or 2,2,6,6-tetramethyl-4-methoxypiperidine-oxyl may also be used in the method according to the invention.

According to the invention, the concentration of the stabilizers in the liquid phase is between 1 ppm and 5000 ppm. Preferably, the concentration is between 5 ppm and 3000 ppm, particularly preferred being a concentration between 10 and 1000 ppm.

The method according to the invention also envisages the injection of oxygen into the distillation column.

The oxygen makes it possible to enhance the efficiency of the stabilizers. It happens, in addition, that it makes it possible to stabilize the gas phase.

The mol ratio between the oxygen injected into the distillation column and the condensed organic vapor at the top of the column is between 0.01% and 1%. Preferably, the mol ratio is between 0.1% and 0.8%, a ratio between 0.1 and 0.5% being particularly preferred. The organic vapor consists, in the methods of purification, substantially of the acrylic monomer. Thus, the mol ratio is calculated relative to the vapor pressure of the acrylic monomer under given temperature and pressure conditions.

The method according to the invention is carried out, in addition, in the presence of a metal sequestering agent. In the context of the invention, there may be used for example: tetraethylenediaminetetraacetic acid (EDTA), trans-1,2-cyclohexanediaminetetraacetic acid (CYDTA), diethylenetraiminepentaacetic acid (DTPA), the pentasodium salt of diethylenetriaminepentaacetic acid (Na₅DTPA), and Na₅DTPA being preferred.

The metal sequestrant is present in the liquid phase in a concentration of between 0.1 ppm and 1000 ppm. Preferably, the sequestrant concentration is between 0.5 ppm and 500 ppm, a concentration between 5 ppm and 100 ppm being particularly preferred.

According to the invention, the simultaneous presence of a stabilizer and a sequestering agent gives rise to a synergistic effect which manifests itself by a stabilization efficiency greater than the sum of the effects attributable to the individual components.

The method according to the present invention is particularly useful on distillations of streams rich in acrylic acid, but it can also be applied with other acrylic monomers.

The invention will be explained in greater detail with the aid of the examples, which are given by way of illustration and without limitation.

### EXAMPLES

The following examples were carried out using a glass assembly simulating a continuous distillation of one of the steps in the purification of acrylic acid.

The assembly consists of a distillation column equipped with a multikit packing made of stainless steel 316, a heating vessel with a thermosiphon, surmounted by a swan neck. The organic vapors are condensed by means of a conventional condenser. A portion of the condensed liquid is recycled at the top of the column after adding liquid phase stabilizers.

The distillation is carried out at a reduced pressure of about 200 mmHg with a temperature of 105° C. in the heating vessel.

The distillation is carried out for 6h, and then the multikit packing is dried and weighed. The stabilizing efficiency of the mixture studied is evaluated by comparing the mass of polymer formed in the packing. For the trials carried out with 4-OH TEMPO, the duration of the trials was set at 3h.

The stream used for all the trials presented consists of 94% crude acrylic acid. This stream continuously feeds the distillation column with a flow rate of 500 g/h. 445 g/h of distillate are continuously drawn off at the top of the column and 75 g/h at the bottom. The flow rate of the organic vapor in the column is 850 g/h. A reflux of 425 g/h is maintained.

The stabilizers used in the examples are abbreviated as follows:

phenothiazine (PTZ),

hydroquinone (HQ),

copper dibutyldithiocarbamate (CB),

4-hydroxytetramethylpiperidine-N-oxyl (4-OH TEMPO), and hydroquinone methyl ether (HQME).

In the different examples, the stabilizers are combined with each other in the proportions indicated in Table 1 below.

The oxygen is injected into the distillation column.

In all the examples, the metal sequestrant is the pentasodium salt of diethylenetriaminepentaacetic acid (Na₅DTPA).

TABLE 1

| Example | Stabilization |
|---|---|
| 1 | 100 ppm PTZ |
| | 8 ppm Na₅DTPA |
| 1A | 100 ppm PTZ |
| 2 | 200 ppm HQ |
| | 8 ppm Na₅DTPA |
| 2A | 200 ppm HQ |
| 3 | 15 ppm CB + 15 ppm HQ |
| | 8 ppm Na₅DTPA |
| 3A | 15 ppm CB + 15 ppm HQ |
| 4 | 15 ppm PTZ + 15 ppm HQ |
| | 8 ppm Na₅DTPA |
| 4A | 15 ppm PTZ + 15 ppm HQ |
| 5 | 10 ppm PTZ + 15 ppm HQ + 5 ppm CB 8 ppm Na₅DTPA |
| 5A | 10 ppm PTZ + 15 ppm HQ + 5 ppm CB |
| 6 | 10 ppm 4-OH TEMPO + 10 ppm HQ |
| | 8 ppm Na₅DTPA |
| 6A | 10 ppm 4-OH TEMPO + 10 ppm HQ |
| 7 | 10 ppm 4-OH TEMPO + 10 ppm HQME |
| | 8 ppm Na₅DTPA |
| 7A | 10 ppm 4-OH TEMPO + 10 ppm HQME |

All the results of the examples are grouped together in Table 2 below.

TABLE 2

| | | |
|---|---|---|
| Example | Duration of the trial | Mass of polymer |
| 1 | 6 h | 5 g |
| 1A | 6 h | 17 g |
| 2 | 6 h | 2 g |
| 2A | 6 h | 15 g |
| 3 | 6 h | 54 g |
| 3A | 3 h* | 66 g |
| 4 | 6 h | 8 g |
| 4A | 6 h | 32 g |
| 5 | 6 h | 17 g |
| 5A | 4 h45* | 74 g |
| 6 | 3 h** | 24 g |
| 6A | 3 h** | 61 g |
| 7 | 3 h** | 2 g |
| 7A | 3 h** | 23 g |

*blocking of the column
**voluntary stopping

These results show that for all the stabilizers or combinations of stabilizers used, the addition of a minimum quantity of Na₅DTPA makes it possible to substantially increase the stabilization of the acrylic acid.

The performance, in terms of prevention of corrosion, of the invention was evaluated by monitoring the loss of weight of stainless steel plates immersed in stabilized and hot acrylic acid.

A glass reactor heated by a jacket is filled with stabilized acrylic acid and kept at 120° C. for 72 hours. The acrylic acid is continuously fed at a flow rate of 120 ml/h, which gives a mean acrylic acid residence time in the reactor of 5 hours.

| | | Nature of the | | |
|---|---|---|---|---|
| | | stainless steel | Mean loss/ | |
| Example | Stabilization | elements | element | |
| 8 | 500 ppm CB + | Plates | 9.4 | mg |
| | 500 ppm HQ + | | | |
| | 8 ppm Na₅DTPA | | | |
| 8A | 500 ppm CB + | Plates | 29.2 | mg |
| | 500 ppm HQ | | | |
| 9 | 500 ppm PTZ + | Coils | 0.9 | mg |
| | 500 ppm HQ + | | | |
| | 8 ppm Na₅DTPA | | | |
| 9A | 500 ppm PTZ + | Coils | 34.8 | mg |
| | 500 ppm HQ | | | |
| 10 | 500 ppm 4-OH | Plates | 1.0 | mg |
| | TEMPO + 500 ppm HQME + | | | |
| | 8 ppm Na₅DTPA | | | |
| 10A | 1000 ppm 4-OH | Plates | 12.0 | mg |
| | TEMPO + 500 ppm HQME + | | | |
| | 8 ppm Na₅DTPA | | | |

The same reator contains stainless steel elements 316L, either in the form of 4 rectangular plates of about 40 cm², or in the form of 2 coils of about 800 cm².

These elements were passivated beforehand. For that, the elements were first of all freed from fat with acetone, and then oxidized with a fluoronitric mixture at 60° C. for 20 min.

Bubbling if a nitrogen stream containing 900 molar ppm of oxygen is also maintained in the reacor.

All these examples and the results obtained are grouped together in the table above.

These examples therefore demonstrate that the addition of a metal sequestrant to the acrylic acid stabilizers makes it possible to substantially reduce the corrosion of stainless stell 316L.

1. A method for stabilizing acrylic monomers in a distillation column, comprising the following steps:
adding at least one stabilizer of acrylic monomers in a total concentration in the liquid phase of between 1 ppm and 5000 ppm;
injecting oxygen into the distillation column with an O₂/organic vapor mol ratio of between 0.01% and 1%;
adding a metal sequestrant in a concentration in the liquid phase of between 0.1 ppm and 1000 ppm.

2. The method as claimed in claim 1, in which the stabilizer is chosen from phenolic derivatives, thiazines, transition metal salts and nitroxide derivatives.

3. The method as claimed in claim 2, in which the stabilizer is chosen from hydroquinone, hydroquinone methyl ether, phenothiazine, methylene blue, copper dibutyldithiocarbamate, manganese acetate, 2,2,6,6-tetramethyl-4-acetoxy-piperidine-oxyl, 2,2,6,6-tetramethyl-4-hydroxypiperidine-oxyl, 2,2,6,6-tetra-methyl-4-methoxypiperidine-oxyl and mixtures thereof.

4. The method as claimed in one of claims 1 to 3, in which the concentration of the stabilizer in the liquid phase is between 5 ppm and 3000 ppm, preferably between 10 and 1000 ppm.

5. The method as claimed in one of claims 1 to 4, in which the mol ratio between the injected oxygen and the organic vapor is between 0.05% and 0.5%, preferably between 0.1 and 0.25%.

6. The method as claimed in one of claims 1 to 5, in which the metal sequestering agent is chosen from tetraethylenediaminetetraaectic acid (EDTA), trans-1,2-cyclohexanediaminetetraacetic acid (CYDTA), diethylene-triaminepentaacetic acid (DTPA), the pentasodium salt of diethylenetriamine-pentaacetic acid (Na₅DTPA), and mixtures thereof.

7. The method as claimed in one of claims 1 to 6, in which the metal sequestrant is present in the liquid phase in a concentration of between 0.5 ppm and 500 ppm, preferably between 5 ppm and 100 ppm.

8. The method as claimed in one of claims 1 to 7, in which the acrylic monomer is chosen from acrylic acid, methacrylic acid, acrylates, methacrylates, acrylonitrile or mixtures thereof.

9. The method as claimed in one of claims 1 to 8, in which at least two stabilizers are used.

10. The method as claimed in one of claims 1 to 9, in which a mixture is used which is chosen from the group: PTZ/HQ/Na₅DTPA, CB/HA/Na₅DTPA, CB/PTZ/HQ/Na₅DTPA, CB/PTZ/HQ/Na₅DTPA and 4-OH-TEMPO/HQME/Na₅DTPA.
